(19) United States
(12) Patent Application Publication
(10) Pub. No.: US 2025/0146856 A1
(43) Pub. Date: May 8, 2025

(54) FLEXIBLE RESERVOIR VOLUME MEASUREMENT SYSTEM AND METHOD

(71) Applicant: Becton, Dickinson and Company, Franklin Lakes, NJ (US)

(72) Inventors: Eric SUN, Waltham, MA (US); Kai QIAN, Wilmington, MA (US)

(73) Assignee: Becton, Dickinson and Company, Franklin Lakes, NJ (US)

(21) Appl. No.: 18/833,640

(22) PCT Filed: Jan. 26, 2023
(86) PCT No.: PCT/US2023/011602

§ 371 Date: Jul. 26, 2024

Related U.S. Application Data
(60) Provisional application No. 63/304,319, filed on Jan. 28, 2022.

(51) Int. Cl. G01F 17/00

(52) U.S. Cl. CPC G01F 17/00

(57) ABSTRACT

Devices, systems, and methods convert linear displacement of expanding and contracting flexible reservoir into rotational displacement which can be correlated to volume of reservoir through numerical measurements. Sensor can comprises one or more components the rotational displacement of which can be correlated to the volume of the flexible reservoir through numerical measurements. Rotation can be converted into digital data indicative of measurement of volume of flexible reservoir. Medical devices for administering liquid drug therapy comprising a flexible reservoir can implement such devices, systems, or methodology.

### FIELD OF DISCLOSURE

Generally, exemplary embodiments of the present disclosure relate to the fields of reservoirs for delivering liquids. More specifically, exemplary embodiments of the present disclosure relate to system and method for measuring and/or monitoring of volume in a flexible medicament reservoir, which can be included in devices and systems for delivering liquid medicinal products, such as insulin.

### BACKGROUND

Medication delivery devices of the present disclosure can be useful in the field of insulin therapy, for example for the treatment of type 1 diabetes. One method of insulin therapy includes syringes and insulin pens that require a needle stick at each injection, typically three to four times per day that are simple to use and relatively low in cost. Another widely adopted and effective method of treatment for managing diabetes is the use of an insulin pump. Insulin pumps can help the user keep blood glucose levels within target ranges based on individual needs, by continuous infusion of insulin.

In the example of medical applications where medication delivery devices of the present disclosure can be particularly useful is patch pumps. A patch pump is an integrated device that facilitates infusion therapy for diabetic patients. A patch pump combines most or all of the fluidic components, including the fluid reservoir, pumping mechanism and mechanism for automatically inserting the cannula, in a single housing which is adhesively attached to an infusion site on the patient's skin, and does not require the use of a separate infusion or tubing set. A patch pump containing insulin adheres to the skin and delivers the insulin over a period of time via an integrated subcutaneous cannula. Some patch pumps may be configured to include wireless communication with a separate controller device, while others are completely self-contained. Such devices are replaced on a frequent basis, such as every three days, particularly when the insulin reservoir is exhausted.

As patch pumps are designed to be a self-contained unit that is worn by the diabetic patient, it is preferable to be as small as possible so that it does not interfere with the activities of the user. Thus, in order to minimize discomfort to the user, it would be preferable to minimize the overall size of the patch pump. Conventional patch pumps or a syringe-type devices typically include a driving mechanism with a single advancing lead screw inside medium or fluid reservoir or chamber to push, advance, or otherwise apply force on the plunger in order to dispense the medium or fluid out of the chamber. In order to minimize the size of the patch pump, its constituent parts should be reduced as much as possible without compromising the accuracy and reliability of device or its feature set. One such part is the reservoir for containing the insulin.

A conventional rigid reservoir, such as a syringe pump, controls the position of a plunger to dispense liquid. A major constraint of such a mechanism is the size of the system because it needs to accommodate both the length of the reservoir and the length of the plunger. Thus, a conventional rigid reservoir can be difficult to deploy in a compact configuration. On the other hand, a flexible reservoir can be efficiently deployed in a compact configuration to further reduce the overall size of a patch pump. An example of such a configuration including a flexible reservoir is disclosed in the U.S. Pat. No. 10,814,062, the entire disclosure of which is incorporated herein by reference.

Volume detection in the reservoir remains an important factor to ensure that the patch pump system functions correctly. Accordingly, there is a need in the art for a medication delivery device and system with a flexible reservoir and volume measurement, which for example may provide accurate fill level data constantly or at a plurality of time points. Such systems and device can also be well suited where constant monitoring and adjustment is necessary, so that many more diabetes patients can benefit from the advantages that patch pump devices provide.

### SUMMARY

Exemplary embodiments of the disclosure may address at least the above problems and/or disadvantages and other disadvantages not described above. Also, exemplary embodiments are not required to overcome the disadvantages described above, and may not overcome any of the problems described above.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

As would be readily appreciated by skilled artisans in the relevant art, while descriptive terms such as “medium”, “medicament”, “stopper”, “plunger”, “thread”, “syringe”, “motor”, “wall”, “top”, “side”, “bottom,” “upper,” “lower,” “proximal”, “distal”, “container”, “reservoir”, “chamber” and others are used throughout this specification to facilitate understanding, it is not intended to limit any components that can be used in combinations or individually to implement various aspects of the embodiments of the present disclosure.

Exemplary embodiments of the present disclosure provide system components that can facilitate a reduction in the overall size or footprint of a drug delivery device, such as a patch pump, by a configuration of a flexible reservoir for dispensing medium or fluid from the reservoir including system and/or components for measuring or monitoring of volume in the flexible reservoir by converting linear displacement into rotational motion.

As would be readily appreciated by skilled artisans in the relevant art, the term “flexible reservoir” includes reservoir structures that are both entirely flexible, such as collapsible fluid pouches or bladders, as well as reservoir structures that are only partially flexible by virtue of having both flexible and rigid wall portions.

Exemplary embodiments of the present disclosure provide devices, systems, and methods that can convert linear displacement of an expanding and contracting flexible reservoir into a rotational displacement which can be correlated to the volume of the reservoir through numerical measurements, where rotation can be converted into digital data indicative of measurement of volume of the flexible reservoir.

Exemplary implementations of embodiments of the present disclosure provide various feature and component which may be deployed individually or in various combinations.

According to an exemplary implementation of the present disclosure, measurement of volume in the flexible reservoir includes a magnetic sensor reading the rotation of a diametrically separated magnetic disc.

Exemplary embodiments of the present disclosure provide a system or a medical device for administering liquid drug therapy to a user with the system, or device, comprising a flexible reservoir for containing a supply of a liquid drug, a sensor for detecting volume in the flexible reservoir, and a controller, monitor, or processor, such as a microprocessor, in communication, such as wired or wireless communication, with the sensor.

According to another exemplary implementation of embodiments of the present disclosure, a sensor comprises one or more components the rotational displacement of which can be correlated to the volume of the flexible reservoir through numerical measurements, for example by the controller, monitor, or processor in communication with the sensor and/or the one or more components.

According to exemplary implementations of embodiments of the present disclosure sensor components can comprise a magnetic disc, or a magnet, and a magnetic rotary sensor, where rotation can be converted into digital data through reading of the rotating diametrically separated magnet with a magnetic rotary sensor.

### BRIEF DESCRIPTION OF THE DRAWINGS

The above and/or other example aspects and advantages will become apparent and more readily appreciated from the following description of example embodiments, taken in conjunction with the accompanying drawings in which:

FIG. 1 diagrammatically illustrates a medication delivery device including volume monitoring according to exemplary embodiments of the present disclosure.

FIG. 2 is a block diagram showing some of the components according to an exemplary implementation of the device of FIG. 1

FIG. 3 illustrates an exploded assembly view of a sensor assembly, according to exemplary embodiments of the present disclosure.

FIG. 4A, 4B, and 4C respectively show perspective side view, cross-sectional view, and top view of an assembled sensor assembly of FIG. 3 according to exemplary implementations of embodiments of the present disclosure.

FIGS. 5A and 5B, respectively show another perspective side view and three-dimensional view of an assemble sensor assembly of FIG. 3, according to exemplary implementation of embodiments of the present disclosure.

FIGS. 6A and 6B, which respectively show yet another perspective side view and three-dimensional view of an assemble sensor assembly of FIG. 3, according to exemplary implementation of embodiments of the present disclosure.

FIG. 7 illustrates an example of a perspective view of a flexible reservoir and sensor assembly implemented in a patch pump system, according to exemplary implementation of embodiments of the present disclosure.

FIG. 8 is a fluidic architecture and metering sub-system diagram of a patch pump system, such as illustrated in FIG. 7, according to exemplary implementation of embodiments of the present disclosure.

### DETAILED DESCRIPTION OF EXEMPLARY EMBODIMENTS

Referring now to the drawings, wherein like reference numerals designate identical or corresponding parts throughout the several views, embodiments of the present disclosure are described as follows.

It will be understood that the terms “include,” “including,” “comprise,” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be further understood that, although the terms “first,” “second,” “third,” etc., may be used herein to describe various elements, components, regions, layers and/or sections, these elements, components, regions, layers and/or sections may not be limited by these terms. These terms are only used to distinguish one element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list. In addition, the terms such as “unit,” “-er (-or),” and “module” described in the specification refer to an element for performing at least one function or operation, and may be implemented in hardware, software, or the combination of hardware and software.

Various terms are used to refer to particular system components. Different companies may refer to a component by different names-this document does not intend to distinguish between components that differ in name but not function.

Matters of these exemplary embodiments that are obvious to those of ordinary skill in the technical field to which these exemplary embodiments pertain may not be described here in detail. In addition, various features of the exemplary embodiments can be implemented individually or in any combination or combinations, and would be understood by one of ordinary skill in the art of medicament delivery devices.

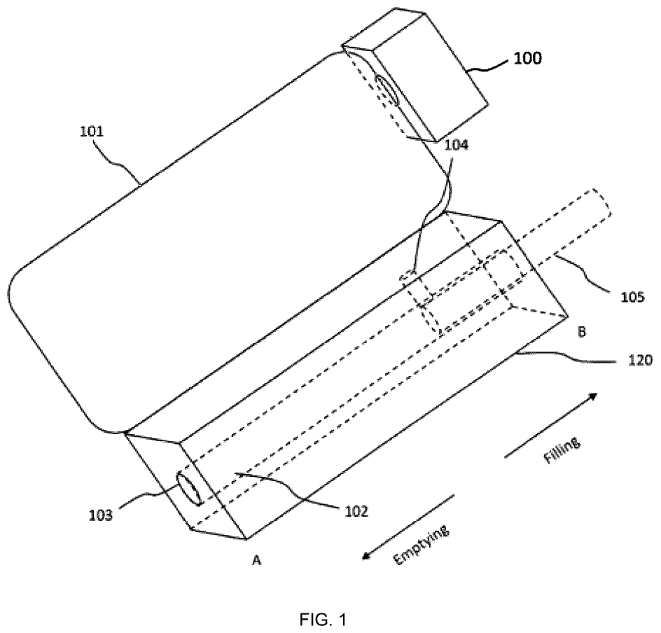

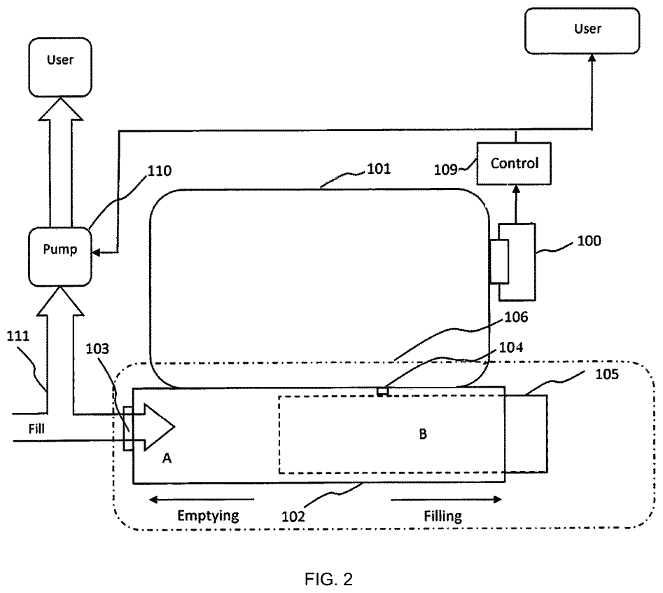

Referring to non-limiting diagrams of FIGS. 1 and 2, a system or device according to exemplary embodiments can comprise a flexible reservoir 101, and pump mechanism 110, for example located downstream of the flexible reservoir 101, the pump mechanism including a plunger 105 disposed within chamber 102 for producing a positive or negative pressure on the fluid inside the chamber 102 and the flexible reservoir 101. In accordance with exemplary embodiments of the present disclosure a sensor assembly or device 100 for measuring volume of a flexible reservoir 101 converts linear displacement of an expanding and contracting flexible reservoir 101 into rotational displacement which can be correlated to the volume of the reservoir 101 through numerical measurements, and rotation can be converted into digital data indicative of measurement of volume of flexible reservoir 101.

In the example of system illustrated in FIGS. 1 and 2, when emptying the flexible reservoir 101, a pump or means can be arranged downstream of the flexible reservoir 101 and the chamber 102, to produce a negative pressure that conveys the fluid from the flexible reservoir 101 to its destination. When filling the flexible reservoir 101, similar means can be arranged to produce a positive pressure that conveys the fluid from another fluid source into the flexible reservoir 101 through the exit/entrance 103. To empty flexible reservoir 101, a pump mechanism 110 produces a negative pressure on the fluid inside the chamber 102 and the flexible reservoir 101, fluid inside the flexible reservoir 101 flows through the side hole 104 into the chamber 102, and through the chamber exit/entrance 103 to conduit 111 to the pump mechanism 110. The negative pressure causes the flexible reservoir 101 to be emptied first and contract. On the other hand, to fill the flexible reservoir 101, a fill syringe or other device can be used to forcibly inject fluid through a valve or seal into the exit/entrance 103. The initial position of the plunger 105 is, for example, position A. The plunger 105 is pushed back, for example to position B, by the positive pressure inside the chamber 102 produced by the filling device. When the plunger 105 passes the side hole 104 in the chamber 102, fluid starts to flow into the flexible reservoir 101 through the side hole 104 and causes the flexible reservoir to he filled and expand.

Sensor assembly 100 converts linear displacement of an expanding and contracting flexible reservoir 101 into rotational displacement which can be correlated to the volume of the reservoir 101 through numerical measurements, and rotation can converted into digital data indicative of measurement of volume of flexible reservoir 101, such that for example control module 109 can then create and/or sends a communication indicative of the volume of the flexible reservoir 101 based on output of sensor assembly 100.

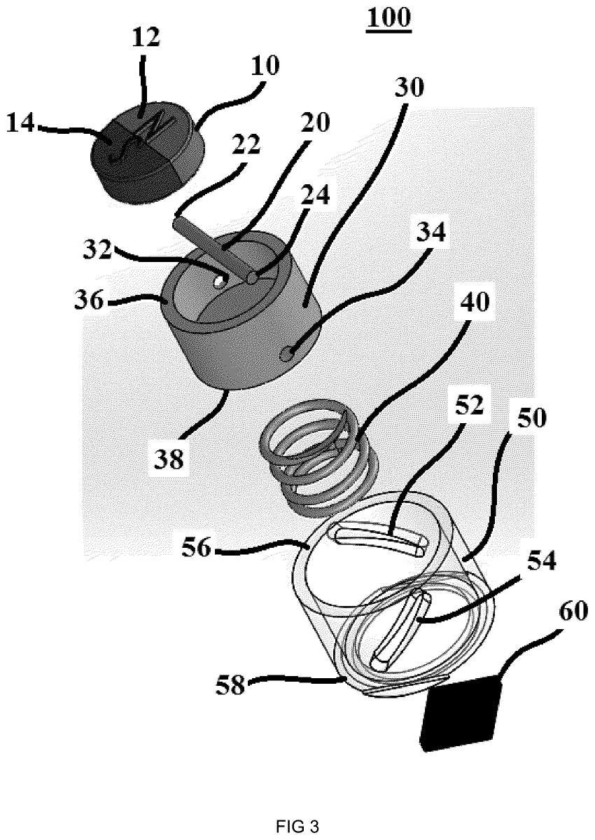

In an exemplary implementation, linear displacement of an expanding and contracting flexible drug reservoir 101 can be converted into rotational displacement through a series of telescoping cylinders and a cam interface that can be provided in sensor assembly 100. Referring to FIGS. 3, a sensor assembly 100 includes a sensor element 10, including a diametrically charged (12, 14) magnet, configured to displace linearly based on expansion and contraction of a flexible reservoir 100, and to rotate due to this linear displacement, for example with respect to a magnetic rotary sensor 60, such as for example a hall effect sensor. Sensor element 10 can be disposed in first telescoping cylinder 30, for example near a distal end 36 of cylinder 30, which is disposed in a second telescoping cylinder 50, where the outer diameter of the first telescoping cylinder 30 is smaller than the inner diameter of the second telescoping element 50, such that for example the second telescoping cylinder 50 surrounds the first telescoping cylinder 30. A biasing element 40, such as a spring, can be provided for axially, or linearly, biasing cylinder 30 and cylinder 50 with respect to each other. Biasing element 40 can be disposed within cylinder 50 between exterior of proximal end 38 of cylinder 30 and interior of proximal end 58 of cylinder 50.

According to exemplary implementation, conversion of axial, or linear, movement into rotational, or circular, movement can be achieved by a mechanical module, such as a cam interface, which can include protrusions 22 and 24 extending radially from exterior surface of cylinder 30. Protrusions 22 and 24 can be integrally formed with cylinder 30, or can be provided by opposite ends of a rod 20 extending through openings 32 and 34 in cylinder 30. Protrusions 22 and 24 interface with respective grooves 52 and 54 of cylinder 50. Grooves 52 and 54 extend diagonally between distal end 56 and proximal end 58 of cylinder 50. When cylinders 30 and 50 move axially, or linearly, with respect to each other, the interaction between protrusions (22 and 24) and respective grooves (52 and 54) results in rotation of cylinders 30 and 50 with respect to each other.

In an exemplary implementation, a diametrically charged (12, 14) magnet can be rotationally and axially (linearly) fixed with respect to cylinder 30, and a hall effect sensor 60 can be rotation and axially (linearly) fixed with respect to cylinder 50. If, for example, cylinder 50 is fixed, or positioned, with respect to reservoir 101, such that expansion and/or contraction of reservoir 101 results in axial movement of cylinder 30 with respect to cylinder 50, then diametrically charged (12, 14) magnet will rotate due to the cam interface and axial displacement of cylinder 30 with respect to cylinder 50 and the magnetic field will fluctuate due to the angular displacement of the magnet, which the hall effect sensor 60 will detect and interpret as sinusoidal voltages, which can be converted into digital data and output indicative of volume of flexible reservoir 101.

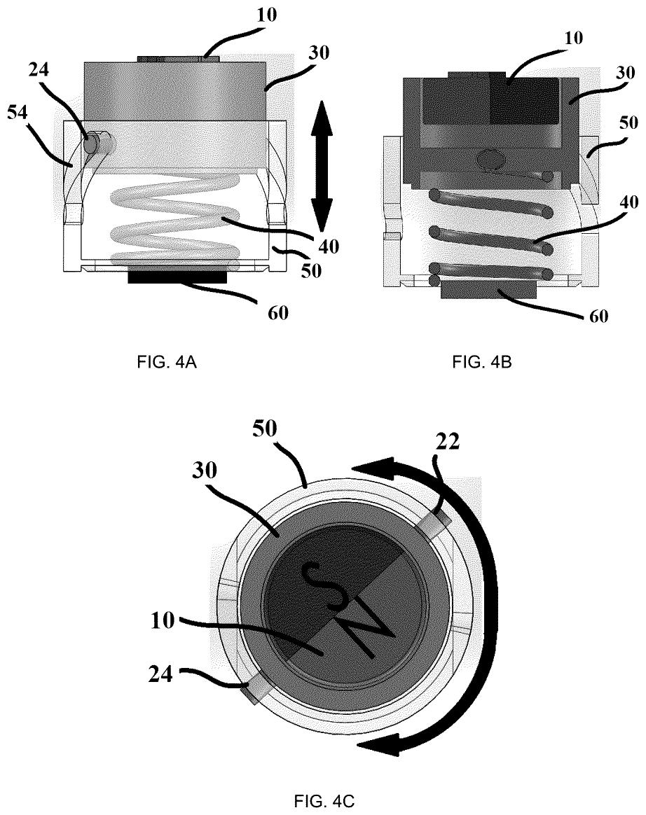

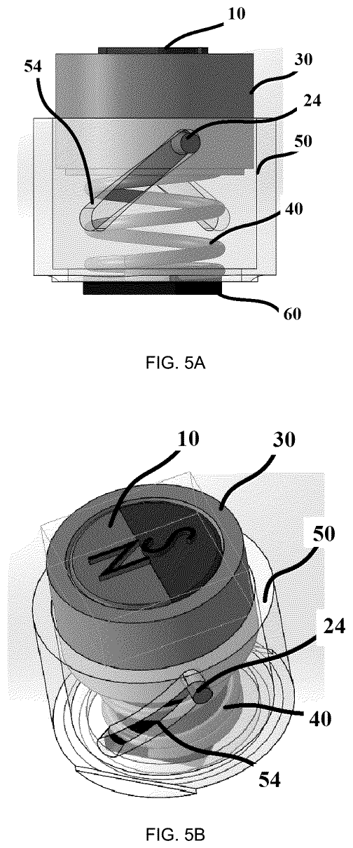

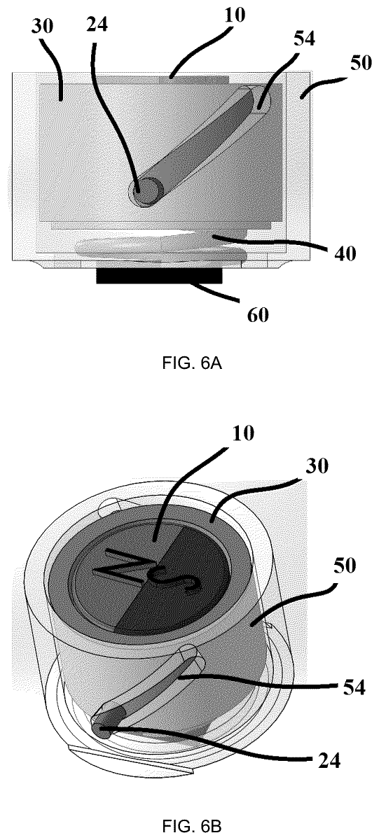

Further detail of a sensor structure according to exemplary implementation of disclosed embodiments are illustrated in the examples of: FIGS. 4A-4C, which respectively show perspective side view, cross-sectional view, and top view of an assemble sensor assembly 100 of FIG. 3; FIGS. 5A and 5B, which respectively show perspective side view and three-dimensional view of an assemble sensor assembly 100 of FIG. 3 when biasing element 40 is extended; and FIGS. 6A and 6B, which respectively show perspective side view and three-dimensional view of an assemble sensor 100 of FIG. 3 when biasing element 40 is compressed.

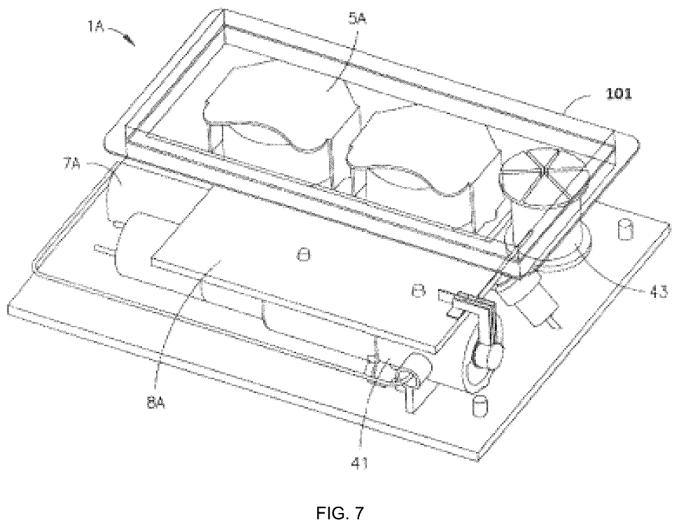

Exemplary embodiments of the present disclosure can be applied to a pump concept, such as for example a patch pump 1A configured to include a flexible reservoir 101, as shown in perspective views of FIG. 7 illustrating patch pump 1A without a cover, with the flexible reservoir 101 (not shown) disposed such that it can fill voids within the patch pump 1A. The patch pump 1A is illustrated with a cannula insertion device 7A that inserts the cannula into the surface of a user's skin. The patch pump 1A can further comprise a power source 5A in the form of batteries; a metering sub-system 41 that monitors the volume of insulin and can includes a volume detecting means 100; control electronics 8A for controlling the components of the device; and a reservoir fill port 43, configured for example for receiving a fill syringe, to fill the reservoir 101.

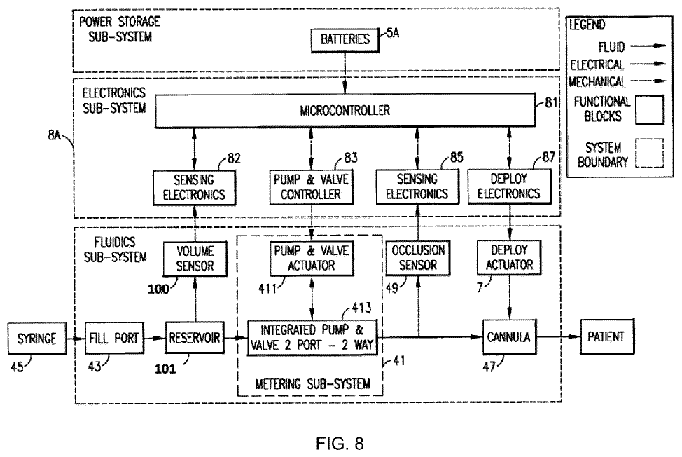

Further, exemplary embodiments of the present disclosure can be applied to a pump concept, such as for example having a fluidic architecture and metering sub-system illustrated in FIG. 8 where control electronics 8A of the patch pump 1A may include a microcontroller 81, sensing electronics 82, pump and valve controller 83, sensing electronics 85 and deployment electronics 87, that control the operation of the patch pump 1A. The patch pump 1A includes a fluidics sub-system that comprises the reservoir 101, a volume sensor assembly 100 for the reservoir 101, and a reservoir fill port 43 for receiving a fill syringe 45 to fill the reservoir 4A. The fluidics sub-system may include a metering system comprising a pump and valve actuator 411 and an integrated pump and valve mechanism 413. The fluidics sub-system may further include an occlusion sensor 49, a deploy actuator or cannula insertion device 7, as well as a cannula 47 for insertion into an infusion site on the user's skin. In exemplary implementations of embodiments of the present disclosure, the signals and/or data to/from sensor assembly 100 can be received, stored, processed, or displayed by microcontroller 81, and can be implemented as part of, or in addition to, sensing electronics 82. For example, the rotational displacement of magnet (12,14) can be correlated to the volume of the reservoir 101 through numerical measurements by microcontroller 81; and/or the reading and conversion of rotation into digital data can be performed by sensing electronics 82 utilizing magnetic disc, or magnet, 10 with magnetic rotary sensor 60.

While the present disclosure has been shown and described with reference to certain exemplary embodiments thereof, it will be understood by those skilled in the art that various changes in form and details may be made therein without departing from the spirit and scope of the embodiments of the present disclosure.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the illustrative embodiments can be easily construed as within the scope of claims exemplified by the illustrative embodiments by programmers skilled in the art to which the illustrative embodiments pertain. Method steps associated with the illustrative embodiments can be performed by one or more programmable processors executing a computer program, code or instructions to perform functions (for example, by operating on input data and/or generating an output). Method steps can also be performed by, and apparatus of the illustrative embodiments can be implemented as, special purpose logic circuitry, for example, an FPGA (field programmable gate array) or an ASIC (application-specific integrated circuit), for example.

The various illustrative logical blocks, modules, and circuits described in connection with the embodiments disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, for example, a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, for example, magnetic, magneto-optical disks, or optical disks. Information carriers suitable for embodying computer program instructions and data include all forms of non-volatile memory, including by way of example, semiconductor memory devices, for example, electrically programmable read-only memory or ROM (EPROM), electrically erasable programmable ROM (EEPROM), flash memory devices, and data storage disks (for example, magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of claims exemplified by the illustrative embodiments. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

Computer-readable non-transitory media includes all types of computer readable media, including magnetic storage media, optical storage media, flash media and solid state storage media. It should be understood that software can be installed in and sold with a central processing unit (CPU) device. Alternatively, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

In addition, the included drawing figures further describe non-limiting examples of implementations of certain exemplary embodiments of the present disclosure and aid in the description of technology associated therewith. Any specific or relative dimensions or measurements provided in the drawings other as noted above are exemplary and not intended to limit the scope or content of the inventive design or methodology as understood by artisans skilled in the relevant field of disclosure.

Other objects, advantages and salient features of the disclosure will become apparent to those skilled in the art from the details provided, which, taken in conjunction with the annexed drawing figures, disclose exemplary embodiments of the disclosure.

1. A system comprising:
a flexible container for a medium;
a sensor assembly configured with respect to the flexible container, wherein volume expansion of the container causes linear displacement of a linearly displaceable sensor component in a first direction and volume contraction of the container causes linear displacement of the linearly displaceable sensor component in a second direction opposite the first direction; and
a module converting the linear displacement of the linearly displaceable sensor component into a measurable rotational displacement correlated to a volume of the container.

2. The system of claim 1, further comprising a microprocessor converting the measurable rotational displacement into digital data format indicative of a measurement of the volume of the flexible container.

3. The system of claim 1, wherein the linearly displaceable sensor component comprises:
a magnet configured to displace linearly in the first direction and the second direction based on the expansion and the contraction of the flexible reservoir;
a mechanical module configured to transfer the linear displacement of the magnet to rotation of the magnet; and
a magnetic sensor rotationally fixed such that the rotation of the magnet with respect to the magnetic sensor causes magnetic field to fluctuate due to the relative angular displacement,
wherein the detected fluctuation in the magnetic field is read as sinusoidal voltages and converted into digital data and output indicative of the volume of the flexible container.

4. The system of claim 3, wherein the magnet comprises a diametrically charged magnet, and the magnetic sensor comprises a Hall effect sensor.

5. The system of claim 3, wherein
the mechanical module comprises:
a first telescoping cylinder having a first exterior diameter, the magnet being rotationally fixed with respect to the first telescoping cylinder, and
a second telescoping cylinder having a second interior diameter; and
the second interior diameter is greater than the first exterior diameter.

6. The system of claim 5, wherein the second telescoping cylinder surrounds the first telescoping cylinder, and the first telescoping cylinder is configured to linearly displace in the first direction and the second direction with respect the second telescoping cylinder based on the expansion and the contraction of the flexible container.

7. The system of claim 5, further comprising a biasing element disposed between the first telescoping cylinder and the second telescoping cylinder to linearly bias the first telescoping cylinder and the second telescoping cylinder.

8. The system of claim 7 wherein the biasing element comprises a spring.

9. The system of claim 5, wherein the mechanical module further comprises an interface converting the linear displacement of the first telescoping cylinder with respect the second telescoping cylinder in the first direction and the second direction into rotational displacement of the first telescoping cylinder with respect to the second telescoping cylinder in respective first rotational direction and second rotational direction.

10. The system of claim 9, wherein the interface comprises a cam interface between the first telescoping module and the second telescoping module.

11. A method of detecting volume of a flexible medicament container, the method comprising:
configured a sensor assembly with respect to a flexible medicament container, such that volume expansion of the flexible medicament container causes linear displacement of a linearly displaceable sensor component in a first direction and volume contraction of the container causes linear displacement of the linearly displaceable sensor component in a second direction opposite the first direction;
converting the linear displacement of the linearly displaceable sensor component into a rotational displacement;
measuring the rotational displacement; and
correlating the measured rotational displacement to a volume of the medicament container.

12. The method of claim 11, further comprising converting the measured rotational displacement into digital data format indicative of a measurement of the volume of the flexible medicament container.

13. The method of claim 11, wherein the linearly displaceable sensor component comprises:
a magnet configured to displace linearly in the first and second directions based on the expansion and the contraction of the flexible reservoir;
a mechanical module configured to transfer the linear displacement of the magnet to rotation of the magnet; and
a magnetic sensor rotationally fixed such that the rotation of the magnet with respect to the magnetic sensor causes magnetic field to fluctuate due to the relative angular displacement,
wherein the measuring of the rotational displacement comprises detecting fluctuation in the magnetic field as sinusoidal voltages, and
the correlating of the measured rotational displacement comprises
converting the measured rotational displacement into digital data, and
generating output indicative of the volume of the flexible medicament container based on the digital data.

14. The method of claim 13, wherein the magnet comprises a diametrically charged magnet, and the magnetic sensor comprises a Hall effect sensor.

15. The method of claim 1, wherein
the mechanical module comprises:
a first telescoping cylinder having a first exterior diameter, the magnet being rotationally fixed with respect to the first telescoping cylinder, and
a second telescoping cylinder having a second interior diameter; and
the second interior diameter is greater than the first exterior diameter.

16. The method of claim 15, wherein the second telescoping cylinder surrounds the first telescoping cylinder, and the first telescoping cylinder is configured to linearly displace in the first direction and the second direction with respect the second telescoping cylinder based on the expansion and the contraction of the medicament container.

17. The method of claim 5, further comprising linearly biasing the first telescoping cylinder and the second telescoping cylinder.

18. The method of claim 17, wherein the mechanical module comprises a biasing element disposed between the first telescoping cylinder and the second telescoping cylinder.

19. The method of claim 15, wherein the mechanical module further comprises an interface converting the linear displacement of the first telescoping cylinder with respect the second telescoping cylinder in the first direction and the second direction into rotational displacement of the first telescoping cylinder with respect to the second telescoping cylinder in respective first rotational direction and second rotational direction.

20. The method of claim 19, wherein the interface comprises a cam interface between the first telescoping module and the second telescoping module.
